(19) United States
(12) Patent Application Publication
(10) Pub. No.: US 2008/0050455 A1
(43) Pub. Date: Feb. 28, 2008

(54) EQUINE GASTRIC ULCER THERAPY COMPOSITIONS AND EQUINE ULCER MANAGEMENT METHOD

(75) Inventor: Larry G. Smith, Washington, PA (US)

Correspondence Address: BLYNN L. SHIDELER;THE BLK LAW GROUP, 3500 BROKKTREE ROAD, SUITE 200, WEXFORD, PA, 15090, US

(73) Assignee: L.C.M. EQUINE NUTRACEUTICALS, INC., Washington, PA (US)

(21) Appl. No.: 11/670,183

(22) Filed: Feb. 1, 2007

Related U.S. Application Data
(60) Provisional application No. 60/764,204, filed on Feb. 1, 2006.

(51) Int. Cl. A61K 36/28; A61K 33/08; A61P 1/04; A61K 33/10

(52) U.S. Cl. 424/687; 424/692; 424/729

(57) ABSTRACT

An equine gastric ulcer therapy composition comprises a pre-packaged, dry feed supplement comprising effective amounts of chamomile tea and an effective amount of slippery elm with effective amounts of apples and a natural sweetener. An alternative composition comprises a pour-able aqueous solution of an effective amount of chamomile tea and an effective amount of slippery elm, wherein the solution has a viscosity of at least 100 cP at about 20° C., or about 80 cSt at about 25° C. A method of treating equine ulcers comprises a regime of Omeprazole for an initial treatment period followed by treatment with the described nutraceutical equine gastric ulcer therapy composition.

### RELATED APPLICATIONS

This application claims the benefit of U.S. Provisional Application Ser. No. 60/764,204 filed Feb. 1, 2006 entitled “Equine Gastric Ulcer Therapy Composition and Method”.

### BACKGROUND OF THE INVENTION

1. Field of the Invention

The present invention relates to equine gastric ulcer therapy compositions and equine gastric ulcer management methods. More particularly the present invention is directed to cost effective yet efficient equine gastric ulcer therapy compositions and equine gastric ulcer management methods.

2. Background Information

Generally colic is a major fear or concern relating to the equine gastric tract. Problems related to the small intestine and large intestine causing colic are well understood and routinely treated. However, the incidence of gastric ulcers is extremely high, particularly in performance horses. The specific percentage of affected horses will vary depending on how the group studied is identified, but the number in any study is alarmingly high. For example, research done in Hong Kong, the USA and including clinical studies in Australia have demonstrated that as many as 80 to 90% active racehorses are victims of gastric ulcers. A study of other performance horses, which are those such as those involved in eventing, showing and western competition, found an equine gastric ulcer rate of over 60%.

In performance horses, there is an association between ulceration and nervousness, but it is unclear whether this is a cause or effect. The performance of racehorses is detrimentally affected by gastric ulcers. Ulcers have been long recognized as a problem in foals, particularly those that are sick for some other reason. It is predicted that most domesticated horses will have ulcers at some point in their lives. This condition may not cause any observable problems in some horses but ulcers can also be both painful and costly. However it is something that can be helped, and possibly even eliminated in some horses with proper management.

The horse's stomach is divided into two parts. The bottom part is glandular that secretes acid and has a protective coating to keep it from being damaged by acid. The top portion of the stomach is designed for mixing of the contents of the stomach. The majority of equine ulcers form in the upper portion of the stomach that is comprised mainly of lining cells similar to those found in the esophagus. This section of the stomach, which serves as a reservoir for ingesta to keep the lower stomach from being overwhelmed, has little protection from the secretions produced in the lower part of the stomach (hydrochloric acid and pepsin) that help to break down ingested food. While the lower glandular section of the stomach has buffers and a mucous lining to help protect it, the whole system is in a delicate balance. This balance can be thrown off by a change in eating habits, feeds, and possibly exercise-induced stresses.

Equine gastric ulcers are, effectively, a man-made or domestication disease. Most horses, allowed the opportunity to free-range graze, rarely have a problem with gastric ulcers, as the equine gastric system evolved to accommodate this type of eating behavior. However, many performance and pleasure horses are not routinely given the opportunity to graze at will or even to eat hay all day. Stall confinement alone can lead to the development of equine gastric ulcers. When horses are fed two times per day, the stomach is subjected to a prolonged period without feed to neutralize the acid. Furthermore, high-grain diets produce volatile fatty acids that can contribute to the development of ulcers. This sets the stage for irritating substances from the lower stomach to invade the upper regions and create problems that can range from heartburn to serious problems.

Research on feeding programs and their effects on ulcers in stall-bound horses have shown that animals with a continual source of hay have significantly lower acid levels in their stomachs. This is to be expected as forage consumption stimulates saliva production and saliva helps to protect the upper region of the stomach. Conversely, horses that have had feed withheld for 24 hours have a much greater level of acid in their stomachs. In a study done in Virginia, bleeding ulcers were induced in horses within three to seven days following a feeding regimen of a 24-hour fast followed by a feed followed by another 24-hour fast. Further, horses in training have very different feeding programs from those allowed the opportunity to graze freely.

Most horses in training are confined for a great portion of the day and are fed large grain meals, a practice which increases the production of gastrin, which is a hormone that stimulates gastric acid. Some studies have also shown that horses produce twice as much saliva when eating hay as they do when eating grain, so grain does not have the beneficial effects of free-choice hay.

Research has also shown that equine gastric ulcers are not caused by Helicobactor pylori bacteria which are a common cause of ulcers in humans. Unlike ulcers in humans, bacteria do not cause equine gastric ulcers. A horse's stomach continually secretes acid, which can result in excess when the horse is not eating regularly due to there being no feed to neutralize the acid. Horses are designed to be grazers with regular intake of roughage. Stress (both environmental and physical) can also increase the likelihood of equine gastric ulcers. Hauling, mixing groups of horses and training can lead to ulcers. Strenuous exercise can decrease both the emptying function of the stomach and blood flow to the stomach, thus contributing to the problem. Even racehorses provided with a constant supply of hay seem more prone to ulcers. The stressful rigors of hard training or travel may indeed be a causative factor in the incidence of gastric ulcers in horses. One study showed that those that were galloped on a treadmill had a much higher incidence of ulcers than horses on the same feeding program that were doing slow trotting work.

Finally, chronic administration of non-steroidal anti-inflammatory drugs such as phenylbutazone can decrease the production of the protective mucus layer, making the stomach more susceptible to ulcers.

Many foals and horses with ulcers show no outward signs, but others may show some symptoms, but there is often no direct correlation in the signs and severity of ulceration. Some performance horses don't show any signs, but do respond to treatment by increasing appetite and condition. An endoscopic examination of the stomach is needed to definitively diagnose gastric ulceration, but few vets have long enough endoscopes to gain access to the stomach of an adult horse so the diagnosis based on clinical signs or response to treatment is often made.

Currently to reduce or prevent gastric ulcers in foals there is an attempt to reduce stresses on the foal, and sick foals that aren't drinking well are placed upon on an existing ulcer treatment to prevent development of ulcers. Providing horses with the opportunity to free-range graze or, failing that, to have access to free-choice hay is believed to be an effective method to keep horses from suffering the effects of acid buildup in the gut. This, however, is not always possible, particularly for horses in heavy training or those that are easy keepers. Time spent eating forage such as grass, chaff and hay seems to be a critical factor.

In stabled horses, it has been suggested that the feeding schedule be altered to mimic a grazing situation by giving the horse access to hay at all times. Recent studies have shown that compared to a grass-hay diet, a diet of lucerne hay and grain provided greater buffering of gastric acidity. Smaller concentrated meals at more frequent intervals, and include chaff to slow down grain intake and increase saliva production, have also been proposed. Reducing grain intake by the addition of calories from fat and maximizing the intake of forage will also help.

Treating equine gastric ulcers involves either inhibiting gastric acid secretion or neutralizing the acid produced. Three classes of drugs can be used to inhibit gastric acid secretion: histamine type-2 antagonists such as cimetidine (sold under the Tagamet® brand name) and ranitidine (as found in the Zantac® or Ulcerguard® brand names); proton pump inhibitors such as omeprazole (as sold under the Gastrogard® and an over the counter version known as Ulcergard™); and prostaglandin analogues. These drugs are believed to be relatively effective in curing equine gastric ulcers in 3 to 4 weeks, but they are also very expensive. A response to treatment will be better when the horse is spelled from work and is in a paddock, but this is often not practical. Lower doses may be effective in preventing recurrence, because if ulcers are treated and the horse stays in work then they usually recur quickly. In addition ranitidine has a 3 day withholding period prior to racing in some jurisdictions. Omeprazole is not always available at all locations, for example it was not previously available in Australia. Further, these drugs can be expensive to administer with Omeprazole being currently about $1200 per month. The cost of this treatment can be prohibitive, and in the case of tight budgeted horse care regimes it necessarily means that this money is simply not available for other areas of the care for the horse.

An alternative to suppression of acid production is to neutralize stomach acid and protect the squamous mucosa from exposure to acid. Antacids may be a good preventative tool and can offer the horse some symptomatic relief of the discomfort associated with ulcers. Human antacids need to be given frequently in large volumes by stomach tube and may have adverse side effects such as inco-ordination, and are simply an impractical solution.

A further promising equine gastric ulcer therapy has used a nutraceutical approach to the problem. In 2001, a therapy regime for equine gastric ulcer therapy was proposed that used routine exposure to chamomile tea and slippery elm bark powder. Specifically ⅓ of a cup of chamomile flowers where boiled in water to make a tea, which was cooled and the slippery elm bark powder was added to the cooled tea. The solution was used to dampen the horse feed twice daily. The composition was shown to provide a mucilaginous lining to the horses' stomach, protecting the mucosa and effectively promoting natural healing of the ulcer. This promising solution held two main drawbacks, first those taking care of the horse will hardly have time to brew, cool, mix the composition and then add it to existing feed twice daily, and second the proposed mixture is not acceptable for equine consumption on a general basis.

Further research studies and related background material on equine gastric ulcers syndrome, symptoms, causes and current treatments can be obtained from Kentucky Equine Research, in Brighton, Victoria Australia (papers by Dr. Joe D. Pagen and Dr. Peter Huntington), Southern Pines Equine Associates (Papers by Dr. Jim Hamilton DVM), www.myHORSEmatters.com (Papers by Frank M. Andrews, DVM, MS, DACVIM, and Scott McClure DVM).

It is the objects of the present invention to address the deficiencies of the prior art equine ulcer treatment options and provide an effective, easy to use equine gastric ulcer treatment composition and method.

### SUMMARY OF THE INVENTION

It is noted that, as used in this specification and the appended claims, the singular forms “a,” “an,” and “the” include plural referents unless expressly and unequivocally limited to one referent.

For the purposes of this specification, unless otherwise indicated, all numbers expressing quantities of ingredients, reaction conditions, and other parameters used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

All numerical ranges herein include all numerical values and ranges of all numerical values within the recited numerical ranges. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The various embodiments and examples of the present invention as presented herein are understood to be illustrative of the present invention and not restrictive thereof and are non-limiting with respect to the scope of the invention.

According to one embodiment of the present invention addressing at least one of the above stated objects, an equine gastric ulcer therapy composition comprising a pre-packaged, dry feed supplement comprising effective amounts of chamomile tea and an effective amount of slippery elm with effective amounts of apples and a natural sweetener. The dry feed composition according to one aspect of the present invention may further include effective amounts of Omega 3 fatty acids from flaxseed oil. The dry feed composition according to one aspect of the present invention may further include effective amounts of licorice root. The dry feed composition according to one aspect of the present invention may further include effective amounts of Milk Thistle. The dry feed composition according to one aspect of the present invention may further include effective amounts of Marshmallow Root. The dry feed composition according to one aspect of the present invention may further include effective amounts of Magnesium Hydroxide, Calcium Carbonate, Glutamic Acid, and Vitamin E.

According to one embodiment of the present invention addressing at least one of the above stated objects, an equine gastric ulcer therapy composition comprising a pre-packaged, pour-able aqueous solution of an effective amount of chamomile tea and an effective amount of slippery elm, wherein the solution has a viscosity of at least 100 cP at about 20° C., or at least 80 cSt at about 25° C.

The term chamomile tea within this application and in reference to the equine gastric ulcer therapy composition references that the chamomile flowers have been placed in an aqueous solution and the temperature raised to at least to a few degrees (e.g. at least within about 10° C.) from a boil, technically this is an infusion. The formation of a “tea” does not require the water brewing the tea to be at a boil, and some teas for human consumption are actually detrimentally affected if brewed at boiling temperature. Chamomile tea is not detrimentally affected if brewed at boiling. There are also “cold” tea brewing techniques, but the term “tea” within this application will reference the elevated temperature of at least a few degrees from boiling.

The term pre-packaged within this application and in reference to the equine gastric ulcer therapy composition references that the composition is preassembled in relative effective amounts. The user may be required to measure out one dosage, but need not combine the constituents. As discussed below, the pre-packaging may be in unit dosages such that measurement by the user is not required.

The term pour-able within this application and in reference to the equine gastric ulcer therapy composition means that the composition may be “poured out” of the packaging for dispensing. Specifically, pour-able refers to a viscosity at ambient temperatures of less than about 250,000 cP.

The phrase aqueous solution within this application and in reference to the equine gastric ulcer therapy composition has the conventional meaning of a water based solution. The term is specifically not intended to exclude other additives. As noted below, thickeners and other nutritional supplements and medicates may be included in the aqueous solution.

The term dry feed within this application in reference to one equine gastric ulcer therapy composition of the invention is as is conventionally known in the art and will reference a powder, granular or even pellet mixture of ingredients with no moisture content other than what may be from ambient conditions.

The phrase effective amount within this application and in reference to the equine gastric ulcer therapy composition refers to a desired unit dosage amount of the associated ingredient times the number of therapy dosages that are in the packaging. The effective amounts per unit dosage of each ingredient are defined hereinafter if not readily understood by those in the equine care fields.

According to one embodiment of the present invention addressing at least one of the above stated objects, a method of treating equine gastric ulcers following the diagnosis of equine gastric ulcers comprises the steps of treating the equine with effective amounts of Omeprazole for an initial treatment period, and, following treatment with Omeprazole, treating the equine with effective amounts of an equine gastric ulcer therapy composition comprising an effective amount of chamomile tea and an effective amount of slippery elm for a period at least as long as the initial treatment period.

The phrase diagnosis of equine gastric ulcers is not intended to be any specific diagnostic procedure, nor intended to suggest involvement of veterinarian personal. It is simply intended to suggest that the equine is suspected of already having ulcers, which is opposed to where a therapy regime is used to minimize the likelihood of occurrence of ulcers, e.g. prevention, where the equine currently likely does not have ulcers.

In one non-limiting aspect of the present invention comprising a pre-packaged, pour-able aqueous solution of an effective amount of chamomile tea and an effective amount of slippery elm, the solution has a viscosity of at least 1000 cP and less than 100,000 cP at about 20° C., and more specifically in one embodiment the solution has a viscosity between 1000 and 25,000 cP at about 20° C. In one embodiment, the solution has a viscosity between 1000 and 7,500 cP at about 20° C., and more specifically the solution has a viscosity between 2000 and 5,000 cP at about 20° C.

In one non-limiting aspect of the present invention comprising a pre-packaged, pour-able aqueous solution of an effective amount of chamomile tea and an effective amount of slippery elm, the solution has a viscosity of at least 100 cSt and less than 15,000 cSt at about 25° C., and more specifically in one embodiment the solution has a viscosity between 100 and 2,000 cSt at about 25° C., and most specifically 100 and 300 cSt at about 25° C.

These and other advantages of the present invention will be clarified in the description of the preferred embodiments taken together with the attached figures.

### BRIEF DESCRIPTION OF THE DRAWINGS

FIG. 1 is a schematic flow chart of a method of making a pre-packaged, pour-able equine gastric ulcer therapy composition according to one aspect of the present invention; and

FIG. 2 is a schematic flow chart of a method of treating an equine gastric ulcer following diagnosis thereof in accordance with one aspect of the present invention.

### DESCRIPTION OF THE PREFERRED EMBODIMENTS

According to one embodiment of the present invention addressing at least one of the above stated objects, an equine gastric ulcer therapy composition comprising a pre-packaged, dry feed supplement comprising effective amounts of chamomile tea and an effective amount of slippery elm with effective amounts of apples and a natural sweetener. The dry feed composition forms an all natural product that promotes healthy equine digestive function. The dry feed supplement will typically be supplied to the equine as a top dress of an effective amount (e.g. about 75 grams) on feed. The term composition here refers effectively to a mixture.

The effective amount of chamomile flowers is 75-120 cc/unit dosage with a unit dosage being about 75 grams for the dry feed version of the present invention. The chamomile flower (or tea) forms 2-50% by weight of the dry feed. The chamomile flowers will generally be in a ground up form but other forms are possible. It is anticipated that the pre-packaged composition will contain multiple dosages, whereby the user will need to measure out, e.g. scoop out, a unit dosage. A unit dosage is about 75 grams and is administered twice daily. The effective amounts can be varied particularly if the treatment regime is changed. For example the effective amounts per unit dosage can be decreased if the daily treatments are increased.

Of course the composition could be pre-packaged in single dosage containers which eliminate the need for the user to even measure, but that is largely a packaging and shipping issue which is based upon whether the single use containers, or sub-containers, are prohibitively expensive. It is expected that the container could also include graduations thereon to assist in the measurement, as known in the dispensing container art, however a single serving size measuring scoop is often the most practical for bulk dry feed compositions.

The effective amount of slippery elm is essentially between a 1-1, 2-1 or even a 3-1 ratio between the slippery elm and the chamomile flowers, with between a 1-1 and 2-1 ratio being more preferred. The slippery elm will typically be in a ground form but other forms are possible. The slippery elm forms 5-70% by weight of the dry feed. The slippery elm amount per dosage can vary with the number of daily treatment regimes desired. Twice daily of about 75 grams each is a preferred dosage regimen.

The natural sweetener may be molasses in a preferred embodiment. Apple and molasses mixtures for equine diets are known. There are a wide variety of mixtures of these ingredients have been proposed. For a general basis ¼ cup of molasses to two apples represents some guidelines for acceptable ratios. The apple/molasses mixture is being utilized to increase palatability to equines. The effective amount of apple/molasses mixture in the ulcer therapy composition according to the invention is 5 to 60% by weight of the dry feed.

The dry feed equine ulcer therapy composition of the present invention further provides a source of Omega 3 fatty acids in the form of flaxseed oil. Studies in humans have found many health benefits with supplementation of omega-3 fatty acids in the diet. While research benefits for horses have not been as numerous, omega-3 supplementation shows potential to provide some healthy results. The effective amount of flaxseed oil in the ulcer therapy composition according to the invention is 0.1 to 25% by weight of the dry feed.

Each molecule of fat or oil consists of three fatty acid molecules and one glycerol molecule. The horse needs a certain amount of fat in its diet, and all fats contain omega-3 and omega-6 fatty acids. Horses require these two types of polyunsaturated fatty acids, the major ones are linoleic acid (an omega-6 fatty acid) and alpha-linolenic acid (an omega-3 fatty acid). These fats are termed essential fatty acids (EFA) because the horse's metabolism can't synthesize them; they must be consumed in the diet to be provided. Although the exact EFA requirements for horses have not been established, they have been demonstrated as necessary for all animals and humans for many normal body functions. Deficiency of EFA in humans and animals includes hair loss, skin problems, and impaired immune function. Pasture grasses and hay, although containing only 2% to 3% fat, have greater concentrations of omega-3 than omega-6 fatty acids. Cereal grains, such as corn and oats, contain much higher levels of omega-6 than omega-3 fatty acids in reference to their total fat content. Both rice bran and soybean oils are higher in omega-3 and lower in omega-6 content than corn oil. And oils from sunflower, flax, and canola seeds contain the largest amount of omega-3's, with higher levels of omega-3 than omega-6 fatty acids. Flaxseed oil is the most concentrated plant source of omega-3 fatty acids or alpha-linoleic acid, also known as ALA. Fish oil is the greatest source of omega-3 fatty acids, with the highest ratio of omega-3: omega-6, and contains the omega-3's eicosapentaenoic acid (EPA) and docosahexanoic acid (DHA). Horses can convert ALA into DHA and EPA in their body tissues, which are used in various physiological functions, so these are not required.

The dry feed equine ulcer therapy composition of the present invention further provides Milk Thistle for its anti-oxidant properties and as an enzyme inhibitor. The effective amount of flaxseed oil in the ulcer therapy composition according to the invention is 0.5 to 20% by weight of the dry feed.

The dry feed equine ulcer therapy composition of the present invention further provides Licorice Root because it promotes the production of a sticky mucous for the reduction of gastric secretion and promotes healing. The Licorice root is in the form of Deglycyrrhizinted Licorice or DGL. The effective amount of DGL in the ulcer therapy composition according to the invention is 0.5 to 20% by weight of the dry feed.

The dry feed equine ulcer therapy composition of the present invention further provides marshmallow root because of its anti-inflammatory and mucilaginous properties. Further it has been shown to have soothing properties and to promote healing. The effective amount of marshmallow root in the ulcer therapy composition according to the invention is 0.5 to 20% by weight of the dry feed.

The dry feed equine ulcer therapy composition of the present invention further provides Magnesium Hydroxide and Calcium Carbonate as acid buffers. The effective amount of Magnesium Hydroxide and Calcium Carbonate in the ulcer therapy composition according to the invention is such that a typical dosage of about 75 grams provides at least 2-2½ grams of calcium and at least 0.1-0.3 grams of magnesium.

The dry feed equine ulcer therapy composition of the present invention further provides Glutamic Acid for proper acidibase balance in the subject. The effective amount of Glutamic Acid in the ulcer therapy composition according to the invention is such that a typical dosage of about 75 grams provides at least 0.1-0.3 grams of Glutamic Acid.

The dry feed equine ulcer therapy composition of the present invention further provides vitamin E as an anti-oxidant and to promote healing. The effective amount of vitamin E in the ulcer therapy composition according to the invention is such that a typical dosage of about 75 grams provides at least 850 IU of Vitamin E.

The present invention, in accordance with one other aspect thereof, provides an equine gastric ulcer therapy composition comprising a pre-packaged, pour-able aqueous solution of an effective amount of chamomile tea and an effective amount of slippery elm, wherein the solution has a viscosity of at least 100 cP at about 20° C., or at least 80 cSt at about 25° C. Further, the present invention provides, in accordance with another aspect thereof, a method of treating equine gastric ulcers following the diagnosis of equine gastric ulcers. The method comprises the steps of treating the equine with effective amounts of Omeprazole for an initial treatment period, and, following treatment with Omeprazole, treating the equine with effective amounts of an equine gastric ulcer therapy composition comprising an effective amount of chamomile tea and an effective amount of slippery elm for a period at least as long as the initial treatment period.

In order to better understand the pour-able equine gastric ulcer therapy composition of the present invention, the term viscosity should be briefly reviewed. Viscosity is, technically, a measure of the resistance of a fluid to deformation under shear stress. It is commonly perceived as “thickness”, or resistance to pouring. Viscosity describes a fluid's internal resistance to flow and may be thought of as a measure of fluid friction. Thus, water and gasoline, at ambient temperatures, are “thin”, having a low viscosity, while vegetable and motor oils are “thick” having a high viscosity. The viscosity of a liquid can change appreciably with a change in the temperature of the liquid, but seldom changes when the pressure is altered.

Viscosity is expressed in “absolute” or “kinematic” terms. The basic unit of absolute viscosity is the “poise” (P), and the common unit for expressing absolute viscosity is the “centipoise” or cP, which is 1/100 of a poise (P). Water at 20.2° C. (68.4° F.) has an absolute viscosity of one centipoise (cP). Kinematic viscosity is different. The basic unit of kinematic viscosity is the “stoke” (St), and the common units for expressing kinematic viscosity is the “Centistoke” (cSt) which is 1/100 of a stoke (St). Kinematic Viscosity and Absolute Viscosity are related as follows:
KINEMATIC VISCOSITY=ABSOLUTE VISCOSITY/SPECIFIC GRAVITY

The following chart may be helpful to demonstrate the relative viscosity values:

| | | |
|---|---|---|
| Hydrogen @ 20° C. | 0.008 | cP |
| Water @ 20° C. | 1.0 | cP |
| Light Machine oil @ 20° C. | 102 | cP |
| Heavy Machine Oil @ 20° C. | 233 | cP |
| Caster Oil@ 20° C. | 983 | cP |
| Glycerin @ 20° C. | 1490 | cP |
| Pancake Syrup @ 20° C. | 2500 | cP |
| Honey @ 20° C. | 10,000 | cP |

The following chart may be helpful to provide a handle on the cSt scale:

| Liquid | cSt @ 25° C. |
|---|---|
| Water | 1 |
| Salad Oil | 50 |
| Maple Syrup | 165 |
| Dish Detergent | 225 |
| Motor Oil (30W) | 225 |
| Glycerin | 550 |
| Corn Syrup (Karo) | 1760 |
| Molasses | 2500 |
| Honey | 15000 |

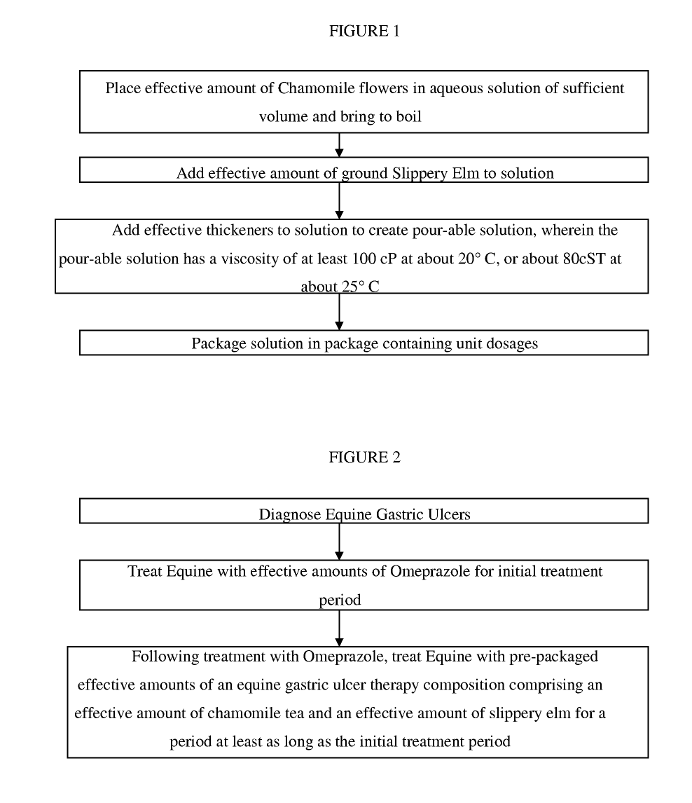

FIG. 1 is a schematic flow chart of a method of making a pre-packaged, pour-able equine gastric ulcer therapy composition according to one aspect of the present invention. The initial step is to place an effective amount of Chamomile flowers in aqueous solution of sufficient volume and bring to boil. As discussed above the mixture need not be elevated to a full boil to form the chamomile tea, but is deemed an effective tool for know when the appropriate temperature has been reached, and boiling does not detrimentally affect the chamomile in the final composition. The cooling of the solution to slightly above ambient (around roughly 25-30° C.) will assure that the chamomile tea has sufficient time to “brew”.

The effective amount of chamomile flowers is 75-120 cc/unit dosage. The chamomile flowers will generally be in a ground up form but other forms are possible. It is anticipated that the pre-packaged composition will contain multiple dosages, whereby the user will need to measure out, e.g. pour out, a unit dosage. Of course the composition could be pre-packaged in single dosage containers to eliminate the need for the user to even measure, but that is largely a packaging and shipping issue upon whether the single use containers (or sub-containers, are prohibitively expensive). It is expected that the container could also include graduations thereon (pour lines) to assist in the measurement, as known in the dispensing container art.

The sufficient volume of water is more variable in the present invention than the unit dosage amounts of chamomile flowers, as the final viscosity can be adjusted with additives (thickeners) as needed. Regardless it is expected that the volume of the aqueous solution forming the chamomile tea is 400 to 2000 cc/unit dosage. The less water, or aqueous solution, is used to form the tea, the less thickening will be required, if at all, however more liquid can provide more efficient brewing. A volume of the aqueous solution forming the chamomile tea of 500 to 1000 cc/unit dosage is a more preferred range.

Following the brewing of the chamomile tea, which will happen as the solution is allowed to cool to only slightly above ambient temperature, the present invention provides for the adding of an effective amount of ground Slippery Elm to solution. There is nothing in the present invention that prevents the addition of the slippery elm prior to the heating of the chamomile flowers in the solution or the agitation of the solution during brewing or other manufacturing assistance known in the art. The effective amount of slippery elm is 75-240 cc/unit dosage, or essentially between a 1-1, 2-1 or even a 3-1 ratio between the slippery elm and the chamomile flowers, with between a 1-1 and 2-1 ratio being more preferred. The slippery elm will typically be in a ground form but other forms are possible.

A significant step in the process of forming the composition in accordance with the present invention if forming the composition in a manner that is most acceptable to the subject horses. Using a relatively large volume of water forming the chamomile tea (per unit dose) will lead, even with the effective amounts of slippery elm, to a very watery substance that has been found to be unsuitable, i.e. too thin).

The present invention adds effective thickeners to solution to create pour-able solution, wherein the pour-able solution has a viscosity of at least 100 cP at about 20° C., or about 80 cST at about 25° C. A number of ranges of solutions have been tried and preferably the solution has a viscosity of at least 1000 cP and less than 100,000 cP at about 20° C., and more specifically in one embodiment the solution has a viscosity between 1000 and 25,000 cP at about 20° C. In one embodiment, the solution has a viscosity between 1000 and 7,500 cP at about 20° C., and more specifically the solution has a viscosity between 2000 and 5,000 cP at about 20° C. Namely, the solution has a viscosity similar to a thin maple syrup. In other embodiments that utilized kinematic viscosity readings, the solution according to one embodiment has a viscosity of at least 100 cSt and less than 15,000 cSt at about 25° C., and more specifically in one embodiment the solution has a viscosity between 100 and 2,000 cSt at about 25° C., and most specifically 100 and 300 cSt at about 25° C.

The thickeners can be any conventional thickeners such as corn starch, or other starches. The composition can include other vitamins, supplements or medicaments, which can replace the thickeners in whole or in part. The preferred supplements and effective amounts are noted in the dry feed composition above. Further, the fluid amount used to brew the chamomile tea may be selected such that when the slippery elm is added, no further thickeners are needed. The amounts of thickeners, if any, are therefore likely to be determined by the efficiencies in the brewing technique. The thickeners may also be used to add flavoring and texture to the composition that is more acceptable or palatable to the horse.

The composition according to the present invention is then packaged in a dispensing container (e.g. 3-Gallon graduated bottle with drain-back pour spout) containing a preset number of unit dosages. The packaging of the present composition is important to the present invention in that it is intended to make it easier on the end user who need not brew the composition according to the above descriptions. The packaging may be a large container filled with separable unit filled containers (e.g. foil pouches), whereby the user can simply empty the entire pouch without measuring unit dosages. The above composition is described in unit dosages amounts. The daily dosages for the present composition are twice daily. This timing makes a package that holds two dosages very convenient for the user who need only dispense ½ container at each application and knows that one package is for one day. There are many possible arrangements for the packaging of the composition according to the present invention, it is simply an important aspect of the present invention that the composition be pre-packaged for the end user.

The composition described in accordance with FIG. 1 is very useful for promoting the self-healing of the equine gastrointestinal tract to heal equine gastric ulcers, and for the prevention of gastric equine ulcers. There may be greater acceptance of the composition of the present invention when used in conjunction with accepted conventional, although expensive, equine gastric ulcer treatments. Specifically, FIG. 2 illustrates a method of treating an equine gastric ulcer following diagnosis thereof in accordance with one aspect of the present invention. The method of treating equine gastric ulcers described in FIG. 2 is following the diagnosis of equine gastric ulcers, as Omeprazole is used primarily after the diagnosis of ulcers. The method comprises the steps of treating the equine with effective amounts of Omeprazole for an initial treatment period of about four weeks. The effective amounts of Omeprazole for treating equine gastric ulcers are well known and the treatment regime need not be elaborated herein other than mentioning that the recommended amounts and applications of Omeprazole will be followed. Following treatment with Omeprazole, the present method provides treating the equine with effective amounts of an equine gastric ulcer therapy composition comprising an effective amount of chamomile tea and an effective amount of slippery elm for a period at least as long as the initial treatment period, and at least about eight weeks. The equine gastric ulcer therapy composition comprising an effective amount of chamomile tea and an effective amount of slippery elm is made as described above in connection with FIG. 1 or in connection with the dry feed version of the invention. It is, of course, possible to overlap the Omeprazole treatment with the treatment of the composition of the present invention, but the overlap does not seem warranted.

The combined treatment technique suggested in FIG. 2 described above may not offer any better actual results than using the composition of the present invention alone. However this duel treatment approach may be well suited for situations where the other changes in the horses training and feeding are being implemented and the Omeprazole treatment will be in effect until these changes take full effect. Further, it simply may be more acceptable to veterinarian professionals that could desire a separation between a healing and a prevention treatment regime. It should be apparent that if the treatment regime using only the composition of FIG. 1 or of the dry feed supplement discussed above is just as effective as the combined treatment of FIG. 2, then the using the composition of FIG. 1 alone will have significant cost advantages over that of FIG. 2.

Whereas particular embodiments of this invention have been described above for purposes of illustration, it will be evident to those skilled in the art that numerous variations of the details of the present invention may be made without departing from the invention as defined in the appended claims. The scope of the present invention is intended to be defined by the appended claims and equivalents thereto.

What is claimed is:

1. An equine gastric ulcer therapy composition comprising a pre-packaged, dry feed supplement comprising effective amounts of chamomile tea and an effective amount of slippery elm with effective amounts of apples and a natural sweetener.

2. The equine gastric ulcer therapy composition of claim 1 wherein the composition further comprises Omega 3 fatty acids.

3. The equine gastric ulcer therapy composition of claim 2 wherein the Omega 3 fatty acids are from flaxseed oil.

4. The equine gastric ulcer therapy composition of claim 1 wherein the composition further includes licorice root.

5. The equine gastric ulcer therapy composition of claim 1 wherein the composition further includes Milk Thistle.

6. The equine gastric ulcer therapy composition of claim 1 wherein the composition further includes Marshmallow Root.

7. The equine gastric ulcer therapy composition of claim 1 wherein the composition further includes Magnesium Hydroxide.

8. The equine gastric ulcer therapy composition of claim 1 wherein the composition further includes Calcium Carbonate.

9. The equine gastric ulcer therapy composition of claim 1 wherein the composition further includes Glutamic Acid.

10. The equine gastric ulcer therapy composition of claim 1 wherein the composition further includes Vitamin E.

11. The equine gastric ulcer therapy composition of claim 1 wherein the natural sweetener is molasses.

12. A method of treating equine gastric ulcers following the diagnosis of equine gastric ulcers comprising the steps of:
Treating the equine with effective amounts of Omeprazole for an initial treatment period; and
Following treatment with Omeprazole, treating the Equine with effective amounts of an equine gastric ulcer therapy composition comprising an effective amount of chamomile tea and an effective amount of slippery elm for a period at least as long as the initial treatment period.

13. The method of claim 12 wherein the treatment with Omeprazole is for about four weeks.

14. The method of claim 12 wherein the treatment with the equine gastric ulcer therapy composition comprising an effective amount of chamomile tea and an effective amount of slippery elm is for a period of at least eight weeks.

15. The method of claim 7 wherein the equine gastric ulcer therapy composition comprising an effective amount of chamomile tea and an effective amount of slippery elm is a pre-packaged, pour-able aqueous solution, wherein the solution has a viscosity of at least 100 cP at about 20° C.

16. The method of claim 7 wherein the equine gastric ulcer therapy composition comprising an effective amount of chamomile tea and an effective amount of slippery elm is a pre-packaged, dry feed supplement.

17. The method of claim 16 wherein the equine gastric ulcer therapy composition includes apples and molasses.

18. The method of claim 16 wherein the equine gastric ulcer therapy composition comprising flaxseed oil, licorice root, Milk Thistle and Marshmallow root.

19. The method of claim 16 wherein the equine gastric ulcer therapy composition comprising Magnesium Hydroxide, Calcium Carbonate, Glutamic Acid and Vitamin E.

20. An equine gastric ulcer therapy composition comprising a pre-packaged, pour-able aqueous solution of an effective amount of chamomile tea and an effective amount of slippery elm, wherein the solution has a viscosity of at least 80 cSt at about 25° C.
